(19) United States
(12) Patent Application Publication
(10) Pub. No.: US 2025/0285742 A1
(43) Pub. Date: Sep. 11, 2025

(54) CLINICAL EVENT MANAGEMENT SYSTEM

(71) Applicant: KONINKLIJKE PHILIPS N.V., Eindhoven (NL)

(72) Inventors: Esther Marjan VAN DER HEIDE, 's-Hertogenbosch (NL); Ron VAN LIESHOUT, Geldrop (NL); Thomas Maria FALCK, Aachen (DE)

(21) Appl. No.: 19/066,217

(22) Filed: Feb. 28, 2025

Related U.S. Application Data
(60) Provisional application No. 63/562,990, filed on Mar. 8, 2024.

(51) Int. Cl. G16H 40/20; A61B 5/00; G08B 21/18

(52) U.S. Cl. CPC G16H 40/20; A61B 5/746; G08B 21/18

(57) ABSTRACT

A method for managing alarm events associated with patients assigned to clinical staff members. The method includes detecting an alarm event associated with a patient clinical status using a patient information system, identifying an assigned clinical staff member, and detecting the staff member's current location using a position tracking system. An availability status of the staff member is determined based on their location. If the staff member is unavailable to attend the alarm event, an alarm event summary is generated and communicated to the staff member using a user output subsystem. The method may involve determining availability based on predetermined areas, detecting non-response or unavailability inputs from the staff member, and communicating summaries to mobile devices.

### FIELD OF THE INVENTION

The present invention relates to a system and method for management and communication of clinical events, such as patient alarms.

### BACKGROUND OF THE INVENTION

An in-patient healthcare unit, such as an intensive care unit (ICU), is a healthcare environment in which patients require continuous monitoring and care. Such units are equipped with a plurality of medical devices configured to continuously track patient vital signs and other clinical parameters, and to provide continuous therapy such as ventilation or infusion. As a result, such an environment can be characterized by frequent alarms and alerts from the monitoring and therapy equipment.

The high volume of alarms in a healthcare unit, such as an ICU, can result in several challenges for healthcare providers. Nurses and other clinical staff may experience alarm fatigue, wherein a multiplicity of alarms at regular intervals can lead to desensitization and potentially missed notifications. Additionally, the noise generated by multiple alarms can create a stressful and distracting environment for both patients and staff, potentially impacting patient recovery and staff performance.

A further challenge in alarm management is the difficulty in prioritizing and responding to multiple simultaneous or closely timed alerts. When a nurse is occupied with one patient, they may not be immediately available to address alarms for other patients under their care. Also, if a nurse is busy outside of the unit or on a break, they may miss important information about one or more of the patients assigned to them. This can result in delayed responses to potentially critical situations or unnecessary interruptions to ongoing patient care activities.

At present, it is necessary for a healthcare worker to manually check symptoms and clinical records of a patient, or speak with colleagues, to understand and be kept informed of what has happened to the patient during the period that they were unable to attend to the situation.

It has been appreciated that a system is needed that overcomes one or more of these problems.

### SUMMARY OF THE INVENTION

The invention is defined by the claims.

There are at least two related and overlapping groups of aspects of the invention. Features pertaining to one aspect group may be applied and incorporated into embodiments of the other aspect group of the invention. The first group of aspects of the invention will now be outlined.

An aspect of the invention is provided a method for managing alarm events associated with each of one or more, wherein each of the one or more patients is assigned to at least one clinical staff member. The method comprises: detecting occurrence of an alarm event associated with a clinical status of a patient using a patient information system; identifying at least one clinical staff member assigned to the patient; detecting a current location of the identified at least one clinical staff member using data from a position tracking system, the position tracking system configured to track a location of the at least one clinical staff member; determining an availability status of the identified at least one clinical staff member based at least in part on the location of the identified clinical staff member; responsive to the availability status indicating that the identified clinical staff member is unavailable to attend to the alarm event:

generating an alarm event summary comprising information relating to the detected alarm event; and communicating the alarm event summary to the identified clinical staff member using a user output subsystem.

This method provides an efficient system for managing alarm events in a clinical setting, ensuring that clinical staff members are informed of important patient events even when they are unavailable to immediately respond. By leveraging location tracking and availability status, embodiments of the invention are able to dynamically determine when to generate and communicate alarm event summaries, reducing alarm fatigue while maintaining patient safety.

With regard to the patient information system, this may for example encompass a real-time patient monitoring and/or therapy network or system and/or a patient medical record system. For example, the patient information system may comprise, or may be communicatively linked with a set of bed-side or point-of-care medical devices. The patient information system may comprise or be communicatively linked with an electronic health record system storing medical information, e.g. medical history information, about each of a set of patients. The patient information system may additionally include any other sources of patient clinical information. With regard to the one or more point-of-care medical devices, these may include patient monitors, ventilators, infusion pumps, and/or any other medical devices that collect and output patient data. These may be configured to output patient clinical information in real time, and this may be collected and recorded by the patient information system.

The alarm event summary may be an alarm event summary report. The exact format of the alarm event summary is not critical. The alarm event summary may in general be a data object comprising data indicative of the information relating to the alarm event. It may comprise a data file containing or comprising said data for example.

In some embodiments, the method comprises determining an availability status indicating that the clinical staff member is available to attend to the alarm event when the clinical staff member is located in at least one predetermined area associated with clinical availability. Additionally or alternatively, the method may comprise determining an availability status indicating that the clinical staff member is unavailable to attend to the alarm event when the clinical staff member is located in at least one predetermined area associated with clinical unavailability.

For example, the area inside of the unit in which a patient is being treated may be classified as an area associated with availability of a staff member, while all areas outside of the unit may be classified as areas associated with unavailability. Areas associated with unavailability may additionally or alternatively include: a medication room, a family consultation room (where family talks take place), break areas, or any other areas where it may be preferable that a staff member is not disturbed.

In some embodiments, the user output subsystem further comprises a user input channel permitting a user to input a response to an alarm event.

The method may comprise detecting non-response of the identified clinical staff member to an alarm event, and responsive to detecting non-response to the alarm event, the method may further comprise generating an alarm event summary for the alarm event and communicating the alarm event summary to the identified clinical staff member using a user output subsystem. By way of example, the method may comprise detecting non-response if the clinical staff member fails to input a response to the alarm within a pre-determined time period following occurrence of the alarm event.

This feature ensures that even when a staff member is initially determined to be available but fails to respond to an alarm event, the system can still generate and communicate an alarm event summary, providing a fallback for situations where staff members may be unexpectedly delayed or unable to respond.

In some embodiments, the method may comprise detecting an input by the identified clinical staff member at the user input channel indicating current unavailability to attend to the alarm event, and responsive to detecting said input, generating an alarm event summary for the alarm event. Additionally or alternatively, the method may comprise detecting an input by the identified clinical staff member at the user input channel indicative of a delegation instruction, and responsive to detecting said input, generating an alarm event summary for the alarm event.

These features provide flexibility for clinical staff members to indicate their unavailability or to delegate tasks, ensuring that the system can adapt to dynamic situations in the healthcare environment and still provide necessary information through alarm event summaries.

Additionally or alternatively, the method may comprise detecting an input by a clinical staff member other than the identified clinical staff member at the user input channel indicating acknowledgment of the alarm event, and responsive to detecting said input, generating an alarm event summary for the alarm event, and optionally communicating this alarm event summary to the identified clinical staff member. This feature provides flexibility for another staff member, other than the identified staff member assigned to the patient, to attend to the alarm in place of the clinical staff member assigned to the patient. In this event, the acknowledgment by the other clinical staff member provides an indication to the system that an alarm event summary should be generated so that the assigned staff member is kept informed of developments with the patient.

In some embodiments, the user output subsystem includes at least one mobile device associated with the identified clinical staff member assigned to the patient. In some embodiments, communicating the alarm event summary to the identified clinical staff member comprises communicating the alarm event summary to the at least one mobile device by means of a mobile communication interface. A mobile device may refer to a mobile computing device, e.g. a smartphone. This feature enables the system to deliver alarm event summaries directly to mobile devices of clinical staff members.

In some embodiments, the alarm event summary includes information relating to clinical actions taken to resolve the alarm event.

In some embodiments, the alarm event summary includes information relating to one or more required clinical actions.

Additionally or alternatively, the alarm event summary may include information relating to the identity of any one or more clinical staff members who attended to the alarm (e.g. to resolve it). It may additionally or alternatively include clinical notes or annotations generated by a clinical staff member who attended to the alarm.

The alarm event summary may also include information relating to a clinical status of the patient at a time of generating the alarm event summary and/or information relating to a clinical status of the patient at a time of communicating the alarm event summary to the clinical staff member.

In some embodiments, the method may comprise detecting multiple alarm events for a same patient during a period when the availability status of the identified at least one clinical staff member assigned to the patient indicates that the at least one clinical staff member is unavailable, and generating a consolidated alarm event summary for the multiple alarm events. This consolidation of multiple alarm events into a single summary improves efficiency of information communication for clinical staff members while still ensuring that all important events are communicated.

In some embodiments, the method further comprises detecting that the identified at least one clinical staff member has entered a predetermined area using data from the position tracking system, and communicating the event summary to the identified at least one clinical staff member in response to detecting that the clinical staff member has entered the predetermined area. This allows for timely delivery of alarm event summaries as soon as a staff member returns to an area where they are likely to be available to address patient needs.

In some embodiments, the method comprises determining, using data from the position tracking system, when the identified clinical staff member is engaged in the care of a patient, and determining the availability status of the clinical staff member as being indicative of unavailability to attend to the alarm event, when the clinical staff member is engaged in the care of another patient. This functionality ensures that staff members are not interrupted while providing care to other patients, maintaining the quality of care while still ensuring important information is communicated at an appropriate time.

The patient information system may be further configured to categorize alarm events based on severity. In some embodiments, the staff scheduling module may be configured to dynamically update assignments of clinical staff to patients based on real-time conditions in the healthcare environment. The event summary module may be configured to generate the alarm event summary in a format customized for the clinical staff member based on the clinical staff member's role or preferences.

A further aspect of the invention is a computer program product comprising computer program code configured, when executed by a processor, to cause the processor to perform a method in accordance with any embodiments described in this disclosure or in accordance with any claim.

A further aspect of the invention is a processing device for managing alarm events associated with each of one or more patients, wherein each of the one or more patients is assigned to at least one clinical staff member. The processing device comprises one or more processors and a communication module. The communication module is communicatively coupled with: a position tracking system configured to track a location of the at least one clinical staff member; and a patient information system configured to detect alarm events associated with a clinical status of each of the one or more patients. The one or more processors are configured to: detect occurrence of an alarm event associated with a patient; identify at least one clinical staff member assigned to the patient; determine an availability status of the identified at least one clinical staff member based at least in part on the location of the identified clinical staff member; responsive to the availability status indicating that the identified clinical staff member is unavailable to attend to the alarm event: generate an alarm event summary comprising information relating to the detected alarm event; and communicate the alarm event summary to the identified clinical staff member using a user output subsystem.

Optionally, the communication module may be further communicatively coupled with a staff scheduling module configured to record assignments of clinical staff members to patients.

Optionally, the communication module may be further communicatively coupled with an event summary module configured to generate alarm event summaries, wherein each alarm event summary comprises information relating to at least one alarm event.

Optionally, the communication module may be further communicatively coupled with a user output subsystem configured to output clinical information to a user by means of one or more user output devices.

A further aspect of the invention is a system for managing alarm events associated with each of one or more patients, wherein each of the one or more patients is assigned to at least one clinical staff member. The system comprises: a position tracking system configured to track a location of the at least one clinical staff member; a patient information system configured to detect alarm events associated with a clinical status of each of the one or more patients; and a processing device in accordance with any embodiment detailed in this disclosure or in accordance with any claim.

Optionally, the system may further comprise a user output subsystem configured to output clinical information to a user by means of one or more user output devices.

Optionally, the system may further comprise a staff scheduling module configured to record assignments of clinical staff members to patients.

Optionally, the system may further comprise an event summary module configured to generate alarm event summaries, wherein each alarm event summary comprises information relating to at least one alarm event.

The second group of aspects of the invention will now be outlined.

An aspect of the invention is a method for managing alarm events associated with each of one or more patients, wherein each of the one or more patients is assigned to at least one clinical staff member. The method comprises: detecting occurrence of an alarm event associated with a clinical status of a patient using a patient information system; identifying at least one clinical staff member assigned to the patient; detecting a current location of the identified at least one clinical staff member using data from a position tracking system, the position tracking system configured to track a location of the at least one clinical staff member; and communicating information relating to the detected alarm event to the at least one clinical staff member using a user output subsystem. The user output subsystem includes a first output channel permitting communication of information related to an alarm event in real time with occurrence of the alarm event, and a second output channel permitting communication of information related to an alarm event at a time subsequent to occurrence of the alarm. Communicating information relating to the detected alarm event comprises selectively outputting the information to the at least one clinical staff member by means of the first output channel or the second output channel in dependence upon the detected current location of the identified clinical staff member.

This method provides an intelligent and adaptive approach to managing alarm events in clinical settings, particularly in intensive care units. By selectively outputting alarm information through different channels based on the staff member's location, the system ensures that critical information reaches the appropriate caregiver at the most suitable time, reducing alarm fatigue and improving patient care efficiency.

The first output channel effectively provides a real-time output channel, while the second output channel effectively provides a post-hoc output channel.

The information relating to the alarm event may comprise an indication of occurrence of the alarm event. For example, this might be output by means of an acoustic alert. For example, output of information relating to the detected alarm event by means of the first output channel may comprise output of an acoustic alert in real time with occurrence of the alarm.

The information relating to the alarm event may additionally or alternatively comprise clinical information relevant to the alarm event. In some embodiments, the method may comprise a step of obtaining clinical information associated with the alarm event using the patient information system. This clinical information may be information relating to patient status.

The information relating to the alarm event may additionally or alternatively comprise information indicative of the clinical event or type of clinical which triggered the alarm.

The user output subsystem may be configured to communicate information to a user by means of one or more user output devices.

In some embodiments, the selective outputting of the information may comprise a step of selecting one or both of the first output channel and second output channel to use to output the information relating to the alarm event based on the location of the clinical staff member. This selection may comprise applying one or more pre-determined rules or algorithms to make the selection based on the detected location of the at least one clinical staff member. For example, there may be a simple rule-based approach, wherein, when the clinical staff member is in a first subset of areas of the healthcare facility, the first output channel is used and when the clinical staff member is in a second subset of areas of the healthcare facility, the second output channel is used and/or both channels are used.

With regard to the patient information system, this may for example encompass a real-time patient monitoring and/or therapy network or system and/or a patient medical record system. For example, the patient information system may comprise, or may be communicatively linked with, a set of bed-side or point-of-care medical devices. The patient information system may comprise or be communicatively linked with an electronic health record system storing medical information, e.g. medical history information, about each of a set of patients. The patient information system may additionally include any other sources of patient clinical information. With regard to the one or more point-of-care medical devices, these may include one or more patient monitors, ventilators, infusion pumps, and/or any other medical devices that collect and output patient data. These may be configured to output patient clinical information in real time, and this may be collected and recorded by the patient information system.

In some embodiments, the selectively outputting the information relating to the detected alarm event comprises determining an availability status of the identified clinical staff member based at least in part on the detected current location of the clinical staff member. In some embodiments, responsive to the availability status indicating that the identified clinical staff member is unavailable to attend to the alarm event, the method comprises selectively outputting the information via the second output channel.

In some embodiments, the method comprises determining an availability status indicating that the clinical staff member is available to attend to the alarm event when the clinical staff member is located in at least one predetermined area associated with clinical availability. Additionally or alternatively, the method may comprise determining an availability status indicating that the clinical staff member is unavailable to attend to the alarm event when the clinical staff member is located in at least one predetermined area associated with clinical unavailability.

For example, the area inside of the unit in which a patient is being treated may be classified as an area associated with availability of a staff member, while all areas outside of the unit may be classified as areas associated with unavailability. Areas associated with unavailability may additionally or alternatively include: a medication room, a family consultation room (where family talks take place), break areas, or any other areas where it may be preferable that a staff member is not disturbed. This feature allows for more nuanced and context-aware determination of staff availability, improving the effective management of alarm communications.

In some embodiments, the user output subsystem further comprises a user input channel permitting a user to input a response to an alarm event.

In some embodiments, the method may comprise detecting non-response of the identified clinical staff member to an alarm event, and responsive to detecting non-response to the alarm event, outputting the information relating to the detected alarm event by means of the second output channel. The method may comprise detecting non-response if no response to the alarm event by the clinical staff member is detected at the user input channel within a pre-determined time period following occurrence of the alarm or following output of information relating to the alarm by the first output channel.

In some embodiments, the method comprises detecting an input by the identified clinical staff member at the user input channel indicating current unavailability to attend to the alarm event, and responsive to detecting said input, further outputting information relating to the alarm event by means of the second output channel.

Preferably, the method further comprises outputting the information relating to the alarm event to a further clinical staff member by means of the first output channel responsive to the user indicating that they are unavailable.

This feature allows staff members to actively manage their availability and ensures that alarms are redirected appropriately when a staff member is unable to respond.

In some embodiments, the method comprises detecting an input by the clinical staff member at the user input channel indicative of a delegation instruction, and responsive to detecting said input, further outputting information relating to the alarm event by means of the second user output channel. In some embodiments, the method may additionally comprise further outputting information relating to the detected alarm event to a further clinical staff member by means of the first user output channel. The further clinical staff member may be identified by the first clinical staff member in the delegation instruction, or may be selected automatically in a selection operation. This feature allows for flexible management of alarm responses, enabling staff members to delegate tasks when necessary.

Additionally or alternatively, the method may comprise detecting an input by a clinical staff member other than the identified clinical staff member at the user input channel indicating acknowledgment of the alarm event, and responsive to detecting said input, further outputting information relating to the alarm event by means of the second user output channel. This feature provides flexibility for another staff member, other than the identified staff member assigned to the patient, to attend to the alarm in place of the clinical staff member assigned to the patient. In this event, the acknowledgment by the other clinical staff member provides an indication to the system that alarm information should be output through the second output channel so that the assigned staff member is kept informed of developments with the patient.

In some embodiments, the output of information relating to the detected alarm event by means of the second output channel may comprise generating an alarm event summary associated with the detected alarm event, the alarm event summary comprising information relating to the detected alarm event, and communicating the alarm event summary to the identified clinical staff member. The alarm event summary may be output to the identified clinical staff member when the staff member's availability status indicates that the staff member is available.

The second output channel may comprise an alarm event summary module configured to generate alarm event summaries, wherein each alarm event summary comprises information relating to at least one alarm event.

In some embodiments, the user output subsystem includes at least one mobile device associated with the identified clinical staff member assigned to the patient. In some embodiments, the output of the alarm event summary to the identified clinical staff member comprises communicating the alarm event summary to the at least one mobile device by means of a mobile communication interface. In some embodiments, the alarm event summary includes information relating to clinical actions taken to resolve the alarm event, and optionally includes information relating to one or more required clinical actions. Additionally or alternatively, the alarm event summary may include information relating to the identity of any one or more clinical staff members who attended to the alarm in order to resolve it. It may additionally or alternatively include clinical notes or annotations generated by a clinical staff member who attended to the alarm.

The alarm event summary may be an alarm event summary report. The exact format of the alarm event summary is not critical. The alarm event summary may in general be a data object comprising data indicative of the information relating to the alarm event. It may comprise a data file containing or comprising said data for example.

In some embodiments, the alarm event summary includes information relating to a clinical status of the patient at a time of generating the alarm event summary and/or information relating to a clinical status of the patient at a time of communicating the alarm event summary to the clinical staff member.

In some embodiments, the method comprises: determining an availability status of the identified at least one clinical staff member based on a current location of the identified clinical staff member, detecting multiple alarm events for a same patient during a period when the availability status of the identified at least one clinical staff member assigned to the patient indicates that the identified at least one clinical staff member is unavailable; and generating a consolidated alarm event summary for the multiple alarm events. This feature helps prevent information overload by consolidating multiple alarms into a single, comprehensive summary.

In some embodiments, the method further comprises: detecting that the identified at least one clinical staff member has entered a predetermined area using data from the position tracking system; and outputting the alarm event summary to the identified at least one clinical staff member by means of the second output channel in response to detecting that the clinical staff member has entered the predetermined area. This feature ensures that staff members receive important alarm information as soon as they enter an appropriate area, improving the timeliness of information delivery.

In some embodiments, the user output subsystem includes at least one mobile device associated with the identified clinical staff member assigned to the patient; and the output of the information relating to the detected alarm event via the first output channel comprises communicating the information to the at least one mobile device associated with the clinical staff member in real time with occurrence of the alarm event.

The position tracking system may comprise a real-time location tracking system.

A further aspect of the invention is a computer program product comprising computer program code configured, when executed by a processor, to cause the processor to perform a method in accordance with any embodiment described in this disclosure or in accordance with any claim.

A further aspect of the invention is a processing device for managing alarm events associated with each of one or more patients, wherein each of the one or more patients is assigned to at least one clinical staff member. The processing device comprises one or more processors and a communication module, wherein the communication module is operatively coupled with the one or more processors. The communication module is communicatively coupled with: a position tracking system configured to track a location of the at least one clinical staff member, a patient information system configured to detect alarm events associated with clinical status of each of the one or more patients, and a user output subsystem configured to output clinical information to a user by means of one or more user output devices. The user output subsystem includes a first output channel permitting communication of information related to an alarm event in real time with occurrence of the alarm event, and a second output channel permitting communication of information related to an alarm event at a time subsequent to occurrence of the alarm event. The one or more processors are configured to: detect occurrence of an alarm event associated with a patient, identify the at least one clinical staff member assigned to the patient, detect a current location of the identified at least one clinical staff member, obtain clinical information associated with the alarm event using the patient information system, and selectively output information relating to the detected alarm event to the identified at least one clinical staff member by means of the first output channel or the second output channel in dependence at least in part upon the detected current location of the identified clinical staff member.

A further aspect of the invention is a system for managing alarm events associated with each of one or more patients is provided, wherein each of the one or more patients is assigned to at least one clinical staff member. The system comprises a position tracking system configured to track a location of the at least one clinical staff member; a patient information system configured to detect alarm events associated with clinical status of each of the one or more patients; and a user output subsystem configured to output clinical information to a user by means of one or more user output devices, wherein the user output subsystem includes a first output channel permitting communication of information related to an alarm event in real time with occurrence of the alarm event, and a second output channel permitting communication of information related to an alarm event at a time subsequent to occurrence of the alarm event. The system further comprises a processing device in accordance with any embodiment described in this disclosure or in accordance with any claim.

Optionally, the system may further comprise a staff scheduling subsystem or module configured to record assignments of clinical staff members to patients. Identifying the at least one clinical staff member assigned to the patient with which the alarm event is associated may be performed using the staff scheduling subsystem.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 shows a flowchart of an example method for managing alarm events in a clinical setting, in accordance with one or more embodiments of the invention;

FIG. 2 shows a block diagram of an example system for managing alarm events in a clinical setting, in accordance with one or more embodiments of the invention;

FIG. 3 shows a flowchart of a further example method for managing alarm events in a clinical setting, in accordance with one or more embodiments of the invention;

FIG. 4 illustrates a block diagram of a further example system for managing alarm events, in accordance with one or more embodiments;

FIG. 5 depicts a further block diagram of an example system for managing alarm events in accordance with one or more embodiments of the invention, the system comprising an alarm module;

FIG. 6 depicts a further block diagram of an example system for managing alarm events in accordance with one or more embodiments of the invention, wherein the system includes a user input channel; and

FIG. 7 illustrates a flowchart of one example implementation of a method for managing alarm events in a clinical setting, in accordance with one or more embodiments of the invention.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

There are at least two related and overlapping groups of aspects of the invention. Features pertaining to one aspect group may be applied and incorporated into embodiments of the other aspect group of the invention. Both pertain to methods and systems for interfacing between patient monitoring and information systems and healthcare workers. Both relate to methods and systems for more efficient input/output between healthcare workers and patient monitoring and information systems. In particular, both relate to methods and systems for more efficient output of patient alarm-related information. For example, both provide methods and system in which a mode or method of the output of alarm-related information is contextually adapted. Both provide methods more particularly in which a mode or method of the output of alarm-related information is contextually adapted based at least in part on a current location of a relevant healthcare worker at a time when an alarm event related to patient they are assigned to occurs. Depending upon the current location of the healthcare worker, the technical steps taken to output (communicate) alarm-related information to the healthcare worker are adapted.

According to a first aspect group of the invention, an availability status of the healthcare worker is determined based at least in part on their current location and, if the healthcare worker availability is classified as being unavailable to attend to an alarm, an alarm event summary report is generated which for example includes details of the clinical event which triggered the alarm and may include steps taken to resolve the alarm (e.g. by other healthcare workers). This can be forwarded or sent to the healthcare worker for their review at a time later than the occurrence of the alarm. This ensures that the patient alarm event can be attended to by colleagues, while still keeping the primary healthcare worker who is assigned to the patient informed of all relevant clinical developments with the patient.

According to a further, overlapping, aspect group of the invention, a more general inventive concept is proposed. According to this aspect group, a user output system is proposed having two channels or modes for outputting information to healthcare workers. One channel or mode is a real-time output channel for communicating an alarm and potentially related clinical information in real time or concurrently with occurrence of the alarm, and a second channel or mode is a post-hoc output channel for communicating the occurrence of an alarm and related clinical information about the alarm after occurrence of the alarm. If an alarm event occurs, the method comprises selectively employing one or both of the first and second output channels or modes based at least in part on a current location of a healthcare worker who is assigned to the patient to which the alarm relates.

The first channel might for example comprise the standard alarm output mechanisms comprised by a patient monitoring system, e.g. including an acoustic alert generated immediately upon occurrence of a clinical event which triggers the alarm. The second channel might be a secondary or supplemental channel which outputs the information in a form that allows the information to be communicated to a user at a time after the alarm, e.g. at a time after the alarm has been resolved or attended to. By way of example, the second channel may comprise a means for generating an alarm event summary report which for example includes details of the clinical event which triggered the alarm and may include steps taken to resolve the alarm (e.g. by other healthcare workers). The second output channel may comprise means for forwarding or sending the report to the healthcare worker for their review at a time later than the occurrence of the alarm. However, this represents just one example implementation. More generally, the second output channel may be employ any of a range of approaches to communicating information about the alarm to a healthcare worked at a later time than the time of occurrence of the alarm. For example, the system may detect when the healthcare worker moved into a different location, or their context changes, and may be configured to communicate the information using one or more output devices located on the unit, e.g. display devices, or using acoustic output devices. The system may for example queue the information for output at a later time, e.g. after the alarm event has been resolved by colleagues.

In accordance with one particularly advantageous set of embodiments, in accordance with the first group of aspects, the system may include one or more of the following features:
A position tracking system: tracking technology utilized by a position tracking system outputs a real-time location of clinical staff, and optionally of medical equipment, and/or patients, for example within a healthcare facility, for example an ICU. This allows for keeping track of where nurses or other clinical staff are located and potentially information about the activity of the clinical staff.
Staff scheduling module: configured to keep track of which staff member is assigned to which patient. For example this comprises a database recording assignments of clinical staff members to patients.
Alarm module: configured to detect and track current and past alarm events.
Event summary module: configured to send a summary of (alarm) events with relevant information to a determined appropriate caregiver (clinical staff member) at a determined appropriate time (as determined by the location of the clinical staff member).
Set of point-of-care devices. These may include for example one or more patient monitors, ventilators, and infusion pumps. These may be configured to generate alarms when changes in condition occur.

In accordance with one or more advantageous embodiments, the system may include one or more of the following features or functions.

In case of an alarm event, the system determines whether the assigned clinical staff member (e.g. nurse) is available and responding to the alarm. If the clinical staff member (e.g. nurse) is not available or responding. for example because the clinical staff member is taking care of another patient, or is outside the care unit (e.g. ICU) the system will send an (alarm) event summary to the clinical staff member as soon as he/she is available, and the alarm is resolved. The summary may provide information for informing the clinical staff member what has been done to resolve the alarm of the patient while not being available.

For example, when a clinical staff member is on a break outside the ICU and the assigned patient has an alarm event, the clinical staff member may automatically receive an alarm event summary upon entering the ICU. In this summary, relevant information about the event are shown, such as who responded, what actions have been taken and what the current situation is.

The above represents just one advantageous set of embodiments, and does not limit the scope of the general inventive principles claimed and outlined further below.

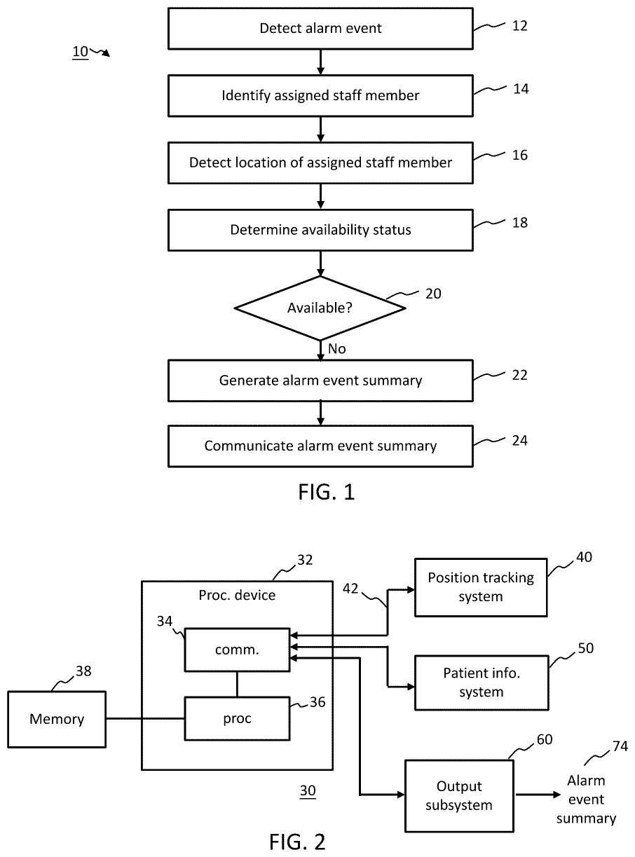

FIG. 1 outlines in block diagram form steps of an example method according to a first group of aspects of the invention. The steps will be recited in summary, before being explained further in the form of example embodiments.

FIG. 1 illustrates a method 10 for managing alarm events in a clinical setting. The alarm events are associated with each of one or more patients. Each of the one or more patients may be assigned to at least one clinical staff member.

The method 10 comprises detecting 12 an alarm event. For example, a patient information system may detect occurrence of an alarm event associated with a clinical status of a patient. The alarm event may be triggered by various factors related to the patient's condition or medical equipment monitoring the patient. For example, the patient information system may detect occurrence of one of a set a predetermined clinical events, and wherein the patient information system is configured to raise an alarm event responsive to detection of any one of these clinical events.

Following the detection of an alarm event, the method 10 comprises identifying 14 at least one clinical staff member assigned to the patient. This identification may be based on staff scheduling information or other assignment data stored in the system. For example, the method may comprise communicating with a staff scheduling module to look up at least one clinical staff member assigned to the patient.

The method 10 further comprises detecting 16 a current location of the identified clinical staff member. This step utilizes data from a position tracking system, which is configured to track the location of clinical staff members, for example locations within a healthcare facility.

After detecting the location of the clinical staff member, the method 10 further comprises determining 18 an availability status of the identified clinical staff member. This determination is based at least in part on the detected location of the clinical staff member. The method may comprise applying one or more pre-determined rules or algorithms for determining or classifying an availability status based on the staff member's location. By way of one non-limiting example, the method may comprise determining an availability status which indicates that the clinical staff member is available to attend to the alarm event when the clinical staff member is located in at least one predetermined area associated with clinical availability. Conversely, the method may comprise determining an availability status which indicates that the clinical staff member is unavailable to attend to the alarm event when the clinical staff member is located in at least one predetermined area associated with clinical unavailability.

The method 10 further comprises a decision step 20 where the availability of the staff member is assessed. The availability status may indicate that the clinical staff member is available to attend to the alarm event. Conversely, the availability status may indicate that the clinical staff member is unavailable to attend to the alarm event.

When the availability status indicates the staff member is not available to attend to an alarm event, the method 10 proceeds to a step of generating 22 an alarm event summary. This summary comprises information relating to the detected alarm event.

The method 10 may further comprise communicating 24 the alarm event summary to the identified clinical staff member. This communication may be performed using a user output subsystem. The user output subsystem may be configured to communicate information to a user by means of one or more user output devices. For example, the user output subsystem may comprise: one or more user output devices, each configured to output clinical information to a user, and a user output controller configured to control output of clinical information to the at least one clinical staff member by means of the one or more user output devices. In a simple example, the alarm event summary is forwarded to a mobile communication device of the clinical staff member, or uploaded to a messaging server so that the staff member can access the alarm event summary when they next check their messaging account.

The proposed method ensures that clinical staff members receive relevant information about alarm events, even when they are not immediately available to respond. This ensures that the clinical staff member is kept informed about the status of the patients to which they are assigned, even when events relating to multiple patients occur at once.

FIG. 2 illustrates a block diagram of an example system 30 for managing alarm events in a healthcare setting, in accordance with the first aspect of the invention. The system 30 is for example configured for implementing the method 10 of FIG. 1.

The system 30 comprises a processing device 32. The processing device 32 comprises a communication module 34 and one or more processors 36. The processing device 32 is connected to a memory 38 for data storage.

The processing device 32 may be configured to interface with one or more subsystems through the communication module 34. These subsystems include a position tracking system 40, which provides tracking data 42 to the processing device 32. The position tracking system 40 is configured to track a location of at least one clinical staff member. The position tracking system 40 may be configured to track a location of each of a plurality of clinical staff members. In some examples, the position tracking system 40 may be implemented as a real-time location tracking system.

The system 30 also includes a patient information system 50 connected to the processing device 32. The patient information system 50 is configured to detect alarm events associated with a clinical status of each of one or more patients. For example, the patient information system may be configured to generate an alarm responsive to detection of any of a set of predetermined clinical events related to patient status.

With regard to the patient information system, this may for example encompass a real-time patient monitoring network or system as well and/or a patient medical record system. For example, the patient information system may comprise, or may be communicatively linked with a set of bed-side or point-of-care medical devices. The patient information system may comprise or be communicatively linked with an electronic health record system storing medical information, e.g. medical history information, about each of a set of patients. The patient information system may additionally include any other sources of patient clinical information. With regard to the one or more point-of-care medical devices, these may include patient monitors, ventilators, infusion pumps, and/or any other medical devices that collect and output patient data. These may be configured to output patient clinical information in real time, and this may be collected and recorded by the patient information system. More details regarding the patient information system will be provided later.

A user output subsystem 60 may also be connected to the processing device 32. The user output subsystem 60 is configured to output information to a user by means of one or more output devices. The one or more output devices may for example include a mobile device carried on the person of the at least one clinical staff member. The one or more output devices may additionally or alternatively include one or more display devices, wherein each display device may be a standalone display device or may be a display device integrated in a point-of-care medical device.

Optionally, the system 30 may further comprise a staff scheduling module (not shown in FIG. 2) configured to record assignments of clinical staff members to patients. The communication module 34 may be communicatively coupled with the staff scheduling module.

Optionally, the system 30 may further include an event summary module configured to generate alarm event summaries, wherein each alarm event summary comprises information relating to at least one alarm event. The communication module 34 may be communicatively coupled with the event summary module.

The processing device 32 may be provided alone as an aspect of the invention, and the system 30 comprising the processing device may be provided as a further aspect of the invention.

The method 10 implemented by the processing device 32 may be embodied as a computer program product comprising computer program code configured to be executed by the one or more processors 36. When executed, this computer program code causes the processing device 32 to perform the steps of the method 10.

With regards to the alarm event summary, this may be an alarm event summary report. The exact format of the alarm event summary is not critical. The alarm event summary may in general be a data object comprising data indicative of the information relating to the alarm event. It may comprise a data file containing or comprising said data for example.

In some embodiments, the user output subsystem 60 may further comprise a user input channel permitting a user to input a response to an alarm event. For example, the system may include one or more user interface devices having user input functionality permitting a user to provide user input and wherein the user interface devices are coupled to the processing device 32 via the user input channel. The one or more user interface devices may include at least one mobile device associated with the at least one clinical staff member, e.g. carried on the person of the clinical staff member.

In some embodiments, the method 10 and system may include functionality for responding to the user input.

In some embodiments, the method 10 may comprise detecting non-response of the identified clinical staff member to an alarm event at the user input channel. By way of example, the method may comprise detecting non-response if no input by the clinical staff member at the user input channel is detected within a pre-determined time period following occurrence of the alarm event. In some embodiments, responsive to detecting non-response to the alarm event, the method 10 may further comprise generating an alarm event summary for the alarm event and communicating the alarm event summary to the identified clinical staff member using a user output subsystem.

In other words, in this set of embodiments, the method 10 may comprise: detecting occurrence of an alarm event, determining an availability status of the identified clinical staff member indicating that the staff member is available to respond to the alarm event, further detecting non-response of the clinical staff member at the user input channel to the alarm event, and, responsive to this, generating the alarm event summary and communicating this to the clinical staff member.

In other words, here, the assigned clinical staff member is initially determined to be available, but if the clinical staff member fails to respond to the alarm event, e.g. within a pre-determined time period, the method may comprise generating and communicating the alarm event summary.

Additionally or alternatively, in some embodiments, the method 10 may comprise detecting an input by the identified clinical staff member at the user input channel indicating current unavailability to attend to the alarm event. Responsive to detecting said input, the method may comprise generating an alarm event summary for the alarm event.

Thus, here, the clinical staff member is able to input a response to the alarm indicating that they are currently unavailable to attend to the alarm event. In other words, in this set of embodiments, the method comprises determining an (initial) availability status indicating availability of the clinical staff member to attend to the alarm event, subsequently detecting from the user input channel an input by the identified clinical staff member indicating unavailability of the staff member to attend to the alarm event, and, responsive to this detection, generating and communicating an alarm event summary to the clinical staff member.

Additionally or alternatively, in some embodiments, the method 10 may comprise detecting an input by the identified clinical staff member at the user input channel indicative of a delegation instruction, and responsive to detecting said input, generating an alarm event summary for the alarm event.

In other words, in this set of embodiments, the method comprises determining an (initial) availability status indicating availability of the clinical staff member to attend to the alarm event, subsequently detecting from the user input channel an input by the identified clinical staff member providing instructions for delegating attendance to the alarm event to another clinical staff member, and, responsive to this detection, generating and communicating an alarm event summary to the clinical staff member.

Optionally, the user input by the identified clinical staff member may include an indication of another clinical staff member to which the alarm event is to be delegated.

Additionally or alternatively, in some embodiments, the method 10 may comprise detecting an input by a clinical staff member other than the identified clinical staff member (who is assigned to the patient) at the user input channel indicating acknowledgment of the alarm event, and responsive to detecting said input, generating an alarm event summary for the alarm event, and optionally communicating this alarm event summary to the identified clinical staff member. This feature provides flexibility for another staff member, other than the identified staff member assigned to the patient, to attend to the alarm in place of the clinical staff member assigned to the patient. In this event, the acknowledgment by the other clinical staff member provides an indication to the system that an alarm event summary should be generated so that the assigned staff member is kept informed of developments with the patient.

The step of detecting an input by a clinical staff member other than the identified clinical staff member may comprise detecting the input at a user interface communicatively linked to the user input channel which is associated with or assigned to said other clinical staff member. For example, the user interface may comprise a software application which the user is logged into with a personal identification, such that the system can detect that the acknowledgement of the alarm has come from a clinical staff member other than the originally identified clinical staff member. However, alternatively, the step of detecting an input by a clinical staff member other than the identified clinical staff member may comprise detecting the input at a user interface communicatively linked to the user input channel which is physically located at a location different to a location of the identified clinical staff member at the time when the user input is detected. This allows the system to infer that the detected acknowledgment of the alarm has not been input by the identified clinical staff member.

In some embodiments, the method may comprise detecting that the identified at least one clinical staff member has entered a predetermined area using data from the position tracking system, and communicating the event summary to the identified at least one clinical staff member in response to detecting that the clinical staff member has entered the predetermined area. For example, the system may detect when the clinical staff member returns to a location associated with clinical availability, e.g. a healthcare unit, and communicate the alarm event summary at this time.

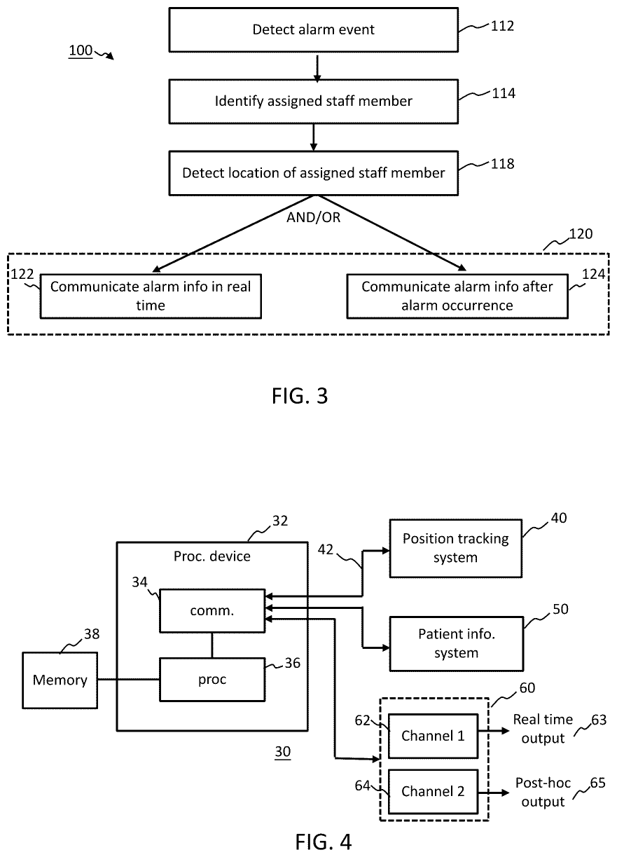

FIG. 3 illustrates an example method 100 in accordance with the second group of aspects of the invention mentioned previously. This provides a further method for managing alarm events in a clinical setting. The alarm events may be associated with each of one or more patients. Each of the one or more of patients may be assigned to at least one clinical staff member.

The method 100 comprises detecting 112 an alarm event. For example, a patient information system may detect occurrence of an alarm event associated with a clinical status of a patient. The alarm event may be triggered by various factors related to the patient's condition or medical equipment monitoring the patient. For example, the patient information system may detect occurrence of one of a set a predetermined clinical events, and wherein the patient information system is configured to raise an alarm event responsive to detection of any one of these clinical events.

Following the detection of an alarm event, the method 100 comprises identifying 114 at least one clinical staff member assigned to the patient. This identification may be based on staff scheduling information or other assignment data stored in the system. For example, the method may comprise communicating with a staff scheduling module to look up at least one clinical staff member assigned to the patient.

The method 100 further comprises detecting 118 the current location of the identified clinical staff. This step utilizes data from a position tracking system, which is configured to track the location of clinical staff members, for example locations within a healthcare facility.

The method further comprises communicating 120 the information relating to the detected alarm event to the at least one clinical staff member using a user output subsystem. As will be explained in more detailed to follow, the user output subsystem includes a first output channel permitting communication of information related to an alarm event in real time with occurrence of the alarm event, and a second output channel permitting communication of clinical information related to an alarm event at a time subsequent to occurrence of the alarm.

The communicating 120 the clinical information comprises selectively outputting information relating to detected alarm event to the at least one clinical staff member using 122 the first output channel and/or using 124 the second output channel in dependence upon the detected current location of the identified clinical staff member.

This may comprise a step of selecting one or both of the first output channel and second output channel to use to output the information relating to the alarm event. This selection may comprise applying one or more pre-determined rules or algorithms to make the selection based on the detected location of the at least one clinical staff member. For example, there may be a simple rule-based approach, wherein, when the clinical staff member is in a first subset of areas of the healthcare facility, the first output channel is used and when the clinical staff member is in a second subset of areas of the healthcare facility, the second output channel is used and/or both channels are used.

In other words, the communicating step 120 step branches into two possible paths, one or both of which are followed selectively in dependence upon a location of the clinical staff member when the alarm event occurs: outputting 122 alarm information using the first output channel and outputting 124 alarm information using second output channel 124. The first output channel 122 may be used for communicating information relating to the detected alarm event in real time, while the second output channel 124 may be used for communicating information relating to the detected alarm event after the alarm occurrence.

The information relating to the alarm event which is communicated may comprise at minimum an indication of occurrence of the alarm event. For example, this might be output by means of an acoustic alert. For example, output of information relating to the detected alarm event by means of the first output channel may comprise output of an acoustic alert in real time with occurrence of the alarm.

The information relating to the alarm event may additionally or alternatively comprise clinical information relevant to the alarm event. In some embodiments, the method may comprise a step of obtaining clinical information associated with the alarm event using the patient information system. This clinical information may be information relating to patient status.

The information relating to the alarm event may additionally or alternatively comprise information indicative of the clinical event or type of clinical event which triggered the alarm.

The method 100 optionally may comprise a step of obtaining clinical information. associated with the detected alarm event for inclusion within the information communicated using the user output subsystem. The obtaining of the clinical information may be performed using the patient information system.

With regards to the user output subsystem, this may be configured to communicate information to a user by means of one or more user output devices. For example, the user output subsystem may comprise one or more user output devices, each configured to output clinical information to a user. The user output subsystem may further comprise a user output controller configured to control output of clinical information to the at least one clinical staff member by means of the one or more user output devices.

The selective outputting 120 may be performed based at least in part on the detected location of the identified clinical staff member. This process may involve determining an availability status of the staff member using their location information. For example, the method may comprise applying one or more pre-determined rules or algorithms for determining or classifying an availability status based on the staff member's location. Thus, in some embodiments, selectively outputting 120 the alarm information may be performed by determining an availability status of the identified clinical staff member based at least in part on the detected current location of the clinical staff member, and selectively outputting the information to one or other or both of the output channels in dependence upon the availability status. For example, responsive to the availability status indicating that the identified clinical staff member is unavailable to attend to the alarm event, the method may comprise selectively outputting 124 the clinical information via the second output channel.

However, in further embodiments, the selection of use of the first output channel, the second output channel or both output channels may be performed without determining an availability status, for example based on applying one or more rules or algorithms to the location information of the at least one clinical staff member.

In some embodiments, the output of information relating to the detected alarm event by means of the second output channel may comprise generating an alarm event summary associated with the detected alarm event, the alarm event summary comprising information relating to the detected alarm event, and communicating the alarm event summary to the identified clinical staff member.

With regards to the alarm event summary, this may be an alarm event summary report. The exact format of the alarm event summary is not critical. The alarm event summary may in general be a data object comprising data indicative of the information relating to the alarm event. It may comprise a data file containing or comprising said data for example.

The user output subsystem may include at least one mobile device associated with the identified clinical staff member assigned to the patient. The method 100 may comprise communicating the alarm event summary to the at least one mobile device by means of a mobile communication interface.

In some implementations, the selective outputting 120 may employ a tiered approach. For example, if a staff member is in a location indicating potential unavailability, the system may first attempt to use the first output channel 122 for urgent alarms. If no response is received within a predetermined time frame, the system may then switch to using the second output channel 124 to deliver an alarm event summary.

The selective outputting 120 may also adapt based on the nature and urgency of the alarm event. For critical alarms, the system may attempt to use both output channels simultaneously, regardless of the staff member's determined location and/or availability status, to ensure the information is communicated as quickly as possible.

By basing the selective outputting 120 on location, the system may optimize the delivery of clinical information, reducing unnecessary interruptions while ensuring that important alerts are communicated effectively to the appropriate staff members.

FIG. 4 shows a block diagram of an example system 30 for managing alarm events in a healthcare setting, in accordance with the second group of aspects of the invention. The system 30 is, for example, configured for implementing the method 100 of FIG. 3. The system of FIG. 4 may be substantially similar to the system of FIG. 2, and therefore like components have been assigned the same reference numeral. The system of FIG. 4 includes a user output subsystem 60 which comprises a first output channel 62 and second output channel 64, as per the discussion above.

By way of summary, the system 30 includes a processing device 32 that comprises a communication module 34, one or more processors 36, and memory 38.

The processing device 32 is communicatively coupled to a number of subsystems. A position tracking system 40 is configured to track a location of the at least one clinical staff member and provides tracking data 42 to the processing device 32. The system 30 further includes a patient information system 50 communicatively connected to the processing device 32. The system 30 includes a user output subsystem 60 comprising a first output channel 62 and a second output channel 64. The first output channel 62 is configured to permit communication of information related to an alarm event in real time with occurrence of the alarm event. The second output channel 64 is configured to permit communication of information related to an alarm event at a time subsequent to occurrence of the alarm event. For example, the first output channel 62 is configured to provide a real time output 63 in real time with occurrence of an alarm event, while the second output channel 64 is configured to provide a post-hoc output 65 subsequent to occurrence of the alarm event.

The one or more processors 36 are configured to detect occurrence of an alarm event associated with a patient using data from the patient information system 50.

With regard to the patient information system 50, this may for example encompass a real-time patient monitoring network or system and optionally also a patient medical record system. For example, the patient information system may comprise, or may be communicatively linked with a set of bed-side or point-of-care medical devices. The patient information system may comprise or be communicatively linked with an electronic health record system storing medical information, e.g. medical history information, about each of a set of patients. The patient information system may additionally include any other sources of patient clinical information. With regard to the one or more point-of-care medical devices, these may include one or more patient monitors, ventilators, infusion pumps, and/or any other medical devices that collect and output patient data. These may be configured to output patient clinical information in real time, and this may be collected and recorded by the patient information system.

In some embodiments, the system 30 may further comprise a staff scheduling module or subsystem (not shown in FIG. 4) configured to record assignments of clinical staff to patients. The step of identifying 114 the at least one clinical staff member assigned to the patient with which the alarm event is associated may be performed using the staff scheduling subsystem.

As noted above, optionally, the output 124 of the information using the second output channel 64 may comprise generating an alarm event summary. In these embodiments, optionally the system may include an event summary module configured to generate alarm event summaries, wherein each alarm event summary comprises information relating to at least one alarm event.

The one or more processors 36 of the processing device 32 are configured to carry out steps of the method of FIG. 3, or any variant thereof.

The processing device 32 may be provided alone as an aspect of the invention, and the system 30 comprising the processing device may be provided as a further aspect of the invention.

The method 100 implemented by the processing device 32 may be embodied as a computer program product comprising computer program code configured to be executed by the one or more processors 36. When executed, this computer program code causes the processing device 32 to perform steps of the method of FIG. 3.

In some embodiments, the user output subsystem 60 may further comprise a user input channel permitting a user to input a response to an alarm event. For example, the system may include one or more user interface devices having user input functionality permitting a user to provide user input and wherein the user interface devices are coupled to the processing device 32 via the user input channel.

In some embodiments, the method 100 may comprise detecting non-response of the identified clinical staff member to an alarm event and, responsive to detecting non-response to the alarm event, outputting the information relating to the detected alarm event by means of the second output channel 64. By way of example, the method 100 may comprise detecting non-response if no input by the clinical staff member at the user input channel is detected within a pre-determined time period following (first) occurrence of the alarm event.

In some examples, the method may comprise detecting non-response if the clinical staff member fails to input a response to the alarm within a pre-determined time period following output of the information relating to the alarm event by the first output channel 62. For example, the method 100 may comprise initially selectively outputting the information associated with the alarm event to the identified clinical staff member via the first output channel 62 (e.g. based on a current location of the clinical staff member being a location associated with clinical availability), detecting non-response of the identified clinical staff member to the information output by the first output channel, and responsive to this detection, outputting the information using the second output channel 64.

Additionally or alternatively, in some embodiments, the method 100 comprises detecting an input by the identified clinical staff member at the user input channel indicating current unavailability to attend to the alarm event, and responsive to detecting said input, further outputting information relating to the detected alarm event by means of the second output channel. For example, the method 100 may comprise initially selectively outputting the information associated with the alarm event to the identified clinical staff member via the first output channel 62, subsequently detecting from the user input channel an input by the identified clinical staff member indicating unavailability of the staff member to attend to the alarm event, and, responsive to this detection, outputting information relating to the detected alarm event via the second output channel 64. The second output channel permits the user to access the information at a later time.

In some embodiments, the method 100 further comprises outputting information relating to the detected alarm event to a further clinical staff member by means of the first output channel 62 responsive to the user indicating that they are unavailable.

Additionally or alternatively, in some embodiments, the method 100 comprises detecting an input by the clinical staff member at the user input channel indicative of a delegation instruction. Responsive to detecting said input, the method 100 may comprise outputting information relating to the detected alarm event by means of the second user output channel 64.

In some embodiments, the method 100 may additionally comprise further outputting information relating to the detected alarm event to a further clinical staff member by means of the first user output channel. The further clinical staff member may be identified by the first clinical staff member in the delegation instruction, or may be selected automatically.

For example, the method 100 may comprise initially selectively outputting the information associated with the alarm event to the identified clinical staff member via the first output channel 62, subsequently detecting from the user input channel an input by the identified clinical staff member providing instructions for delegating attendance to the alarm event to another clinical staff member, and, responsive to this detection, outputting the information relating to the detected alarm event by means of the second user output channel 64. This thereby allows the identified clinical staff member to access the information at a later time.

Additionally or alternatively, in some embodiments, the method 100 may comprise detecting an input by a clinical staff member other than the identified clinical staff member at the user input channel indicating acknowledgment of the alarm event, and responsive to detecting said input, further outputting information relating to the alarm event by means of the second user output channel 64. This feature provides flexibility for another staff member, other than the identified staff member assigned to the patient, to attend to the alarm in place of the clinical staff member assigned to the patient. In this event, the acknowledgment by the other clinical staff member provides an indication to the system that alarm information should be output through the second output channel 64 so that the assigned staff member is kept informed of developments with the patient.

The step of detecting an input by a clinical staff member other than the identified clinical staff member may comprise detecting the input at a user interface communicatively linked to the user input channel which is associated with or assigned to said other clinical staff member. For example, the user interface may comprise a software application which the user is logged into with a personal identification, such that the system can detect that the acknowledgement of the alarm has come from a clinical staff member other than the originally identified clinical staff member. However, alternatively, the step of detecting an input by a clinical staff member other than the identified clinical staff member may comprise detecting the input at a user interface communicatively linked to the user input channel which is physically located at a location different to a location of the identified clinical staff member at the time when the user input is detected. This allows the system to infer that the detected acknowledgment of the alarm has not been input by the identified clinical staff member.

In some embodiments, the method may comprise detecting that the identified at least one clinical staff member has entered a predetermined area using data from the position tracking system; and outputting the information relating to the detected alarm event to the identified at least one clinical staff member by means of the second output channel in response to detecting that the clinical staff member has entered the predetermined area. For example, the system may detect when the clinical staff member returns to a location associated with clinical availability, e.g. a healthcare unit, and communicate the alarm information at this time.

In the description above, one example implementation of the second output channel 64 is proposed, wherein output of information by the second output channel comprises communicating information relating to the alarm event by generating and communicating an alarm event summary. However, the second output channel 64 may implement various alternative methods for communicating alarm event information to clinical staff members when they are unavailable or unable to respond immediately. One option might be to employ a visual notification system. The second output channel may integrate with one or more visual output devices, such as one or more display devices, which may be controlled to display information related to a detected alarm event upon return of the clinical staff member to a location associated with clinical availability. Another option may be to communicate alarm information by means of wearable device notifications: The system may send haptic or visual notifications to wearable devices worn by the clinical staff member once the clinical staff member moves back to an area associated with clinical availability, or otherwise becomes available again. Another option may be to use an automated text-to-speech output device, for example worn in the clinical staff member's car, and to output the information about the detected alarm using this output device as soon as the staff member becomes available again.

There will now be described implementation details which are applicable to both the first aspect of the invention (FIG. 1 and FIG. 2) and the second aspect of the invention (FIG. 3 and FIG. 4). Therefore any features, embodiments or implementation options discussed below may be understood as applicable to either or both of the method and system of FIG. 1-FIG. 2 and the method and system of FIG. 3-FIG. 4.

In some embodiments, the tracking of the at least one clinical staff member may comprise tracking, by means of a position tracking system, a position of at least one tracking device associated with a clinical staff member. The tracking device may be carried on the person of the clinical staff member. In some embodiments, the tracking device may be provided by a mobile computing device, for example a smartphone device. In other words, the system may comprise at least one mobile computing device carried on the person of the at least one clinical staff member, and wherein the tracking of the at least one clinical staff member comprises tracking a position of the mobile computing device carried by the at least one clinical staff member.

The position tracking system may utilize any of various technologies such as Wi-Fi triangulation, radio sensor technology, use of RFID tags, or camera-based tracking technology to determine and track the location of clinical staff within a healthcare facility.

By way of example, one means for implementing the position tracking technology is by use of real-time location system (RTLS) technologies to track the location of clinical staff members within a healthcare facility. In some embodiments, the position tracking subsystem 40 may utilize an infrared (IR) based system for indoor positioning.

In implementations using an IR-based tracking system, clinical staff members may wear badges equipped with IR receivers. These badges may be designed to be lightweight and unobtrusive, allowing staff to carry them comfortably throughout their shifts. A system of IR beacons may be mounted at a series of locations in the healthcare facility. These locations may include for example patient rooms, nursing stations, medication rooms, ICU units, and other areas relevant to patient care and staff activities.

The IR beacons may be configured to broadcast a unique Location ID corresponding to their specific position within the facility. As clinical staff members move through the facility, their badges may receive these Location ID broadcasts from nearby IR beacons. Upon receiving a Location ID, the badge may combine this information with its own unique Badge ID and transmit this data package to a central RTLS server using radio frequency (RF) communication.

The RF communication used for transmitting the combined Badge ID and Location ID information may be implemented using various wireless technologies. In some cases, the system may utilize a 900 MHz frequency band for this communication. Alternatively, the system may leverage existing Wi-Fi infrastructure within the healthcare facility or employ Bluetooth technology for data transmission.

The central RTLS server may receive and process the data transmitted by the badges, allowing the system to maintain real-time tracking of each staff member's location. This location data may then be communicated to the processing device 32.

The position tracking system 40 may encompass the central RTLS server, the IR beacons and the IR receivers carried by the clinical staff members. Of course, the IR receivers could be integrated in forms other than badges, example other wearable units, e.g. neck-worn units or head-worn units, or integrated in clothing.

Additionally or alternatively to the IR-based system, Near Field Communication (NFC) technology may be used for precise location tracking at specific checkpoints or stations within the facility. Bluetooth-based positioning, particularly using Bluetooth Low Energy (BLE) beacons, may offer another alternative for indoor location tracking.

With regard to the patient information system 50, this may for example encompass a real-time patient monitoring and/or therapy network or system as well as a patient medical record system. For example, the patient information system 50 may comprise, or may be communicatively linked with, a plurality of point-of-care devices. These point-of-care devices may include one or more patient monitors, ventilators, infusion pumps, and/or other medical devices that collect and transmit patient data. The point-of-care medical devices may be configured to output patient clinical information in real time, and this may be collected and recorded by the patient information system.

The patient information system may comprise or be communicatively linked with an electronic health record system storing medical information, e.g. medical history information, about each of a set of patients. The patient information system may additionally include any other sources of patient clinical information.

The one or more patient monitors connected to the patient information system 50 may acquire and transmit real-time physiological data related to patients. This data may include vital signs such as heart rate, blood pressure, respiratory rate, and oxygen saturation. The one or more ventilators may output information about patient respiratory status, including tidal volume, respiratory rate, and airway pressure. The one or more infusion pumps may transmit data related to medication administration, including drug types, dosages, and infusion rates.

The point-of-care devices contribute to the real-time acquisition of patient monitoring information, which is then made available through the patient information system 50.

The patient information system 50 may be configured to exchange information with the processing device 32, for example through the communication module 34. This information exchange allows the processing device 32 to retrieve relevant patient data and send updates or queries back to the patient information system 50.

As noted above, in some embodiments, the patient information system is configured to generate alarms or alerts responsive to occurrence of any one or more pre-determined clinical events associated with a clinical status of a patient, or associated with a planned care workflow for a patient. These alarms or events may be raised by the patient information system and may be forwarded to the processing device 32, for example via the communication module 34.

As noted above, some embodiments involve determining an availability status of the identified at least one clinical staff member based at least in part on the location of the identified clinical staff member.

In accordance with one or more embodiments, the system may define predetermined areas within the healthcare facility that are associated with different levels of availability. For example, areas within the patient care unit may be designated as zones where staff members are considered available, while areas such as break rooms, medication rooms, or locations outside the unit may be designated as zones where staff members are potentially unavailable. A step of determining an availability status of the identified at least one clinical staff member may comprise first evaluating the staff member's current location against these predefined zones. If the clinical staff member is determined to be currently located in at least one predetermined area associated with clinical availability, the method may comprise determining an availability status for the clinical staff member indicating that the clinical staff member is available to attend to the alarm event. Conversely, if the at least one clinical staff member is determined to be currently located in at least one predetermined area associated with clinical unavailability, the method may comprise determining an availability status for the clinical staff member indicating that the clinical staff member is unavailable to attend to the alarm event.

In some embodiments, the availability status determination may also take into account additional factors beyond just location. For instance, the system may consider the duration of time the staff member has been in a particular location, their current assigned tasks, or any manually input status updates provided by the staff member.

For example, in some embodiments, the method may comprise determining, using data from the position tracking system, when the identified clinical staff member is engaged in the care of a patient, and determining the availability status of the clinical staff member as being indicative of unavailability to attend to the alarm event when the clinical staff member is engaged in the care of another patient.

As noted above, some embodiments involve generating and communicating one or more alarm event summaries associated with alarm events.

Each alarm event summary may include information relating to clinical actions taken to resolve a detected alarm event, for example in the absence of the at least one clinical staff member assigned to the patient to which the alarm event relates. For example, if the clinical staff member is not able to attend to an alarm event, and the event is resolved in their absence, the alarm event summary may provide a summary of clinical information relevant to the resolving of the alarm event. This information might be obtained automatically based on monitoring of one or more medical devices associated with the patient, for example one or more patient monitors or one or more therapy devices such as ventilators and/or infusion devices. From monitoring status data output by these devices, actions taken to resolve the alarm event might be automatically inferred. Alternatively, information relating to clinical actions taken to resolve the alarm event might be input manually by one or more users of the system, for example by one or more clinical staff members different from the identified clinical staff member assigned to the patient.

Optionally each alarm event summary may include information relating to one or more required clinical actions still to be performed. For example, an alarm event or a change in clinical status may give rise to a need to perform one or more subsequent clinical actions such as administering medication or changing a medication dose. This information might be inferred for example based on one or more of: information about the alarm event, information about clinical actions already taken, information about a current clinical status of the patient, information about a care protocol or pathway which the patient is being treated with, and/or information about a pre-determined care workflow associated with the patient. In some embodiments, it may be determined based on user input by one or more users, for example by one or more clinical staff members.

The alarm event summary 74 may include information relating to clinical actions taken to resolve the alarm event. The alarm event summary 74 may also include information relating to the identity of clinical staff members who attended to the alarm to resolve it. This allows the assigned clinical staff member to know who was involved in addressing the alarm event.

Additionally or alternatively, the alarm event summary 74 may include information relating to a clinical status of the patient at a time of generating the alarm event summary and/or at a time of communicating the alarm event summary to the clinical staff member. This ensures that the clinical staff member receives up-to-date information about the patient's condition.

The alarm event summary 74 may include clinical notes or annotations generated by a clinical staff member who attended to the alarm. These notes provide additional context and insights about the alarm event and the actions taken.

The alarm event summary may be an alarm event summary report.

The exact format of the alarm event summary is not critical. The alarm event summary may in general be a data object comprising data indicative of the information relating to the alarm event. It may comprise a data file containing or comprising said data for example. Such a data object or data file may be provided in a standardized universal file format, such as a PDF file, a text file, a spreadsheet file etc. Alternatively, it may be provided in a proprietary file format which is readable by components of the system. It may take the form of a written document, e.g. a report document. In other words, the information may be represented in natural language form. It may however take a different form, wherein the information is represented or encoded in a machine-readable form, and wherein communication of the information is achieved by reading of the alarm event summary at a client user interface device and presenting the information to the user in a human-readable form.

The method may generate a consolidated alarm event summary for multiple alarm events. This consolidation may occur in the event that there are detected multiple alarm events for a same patient during a period when the availability status of the identified at least one clinical staff member assigned to the patient indicates that the at least one clinical staff member is unavailable.

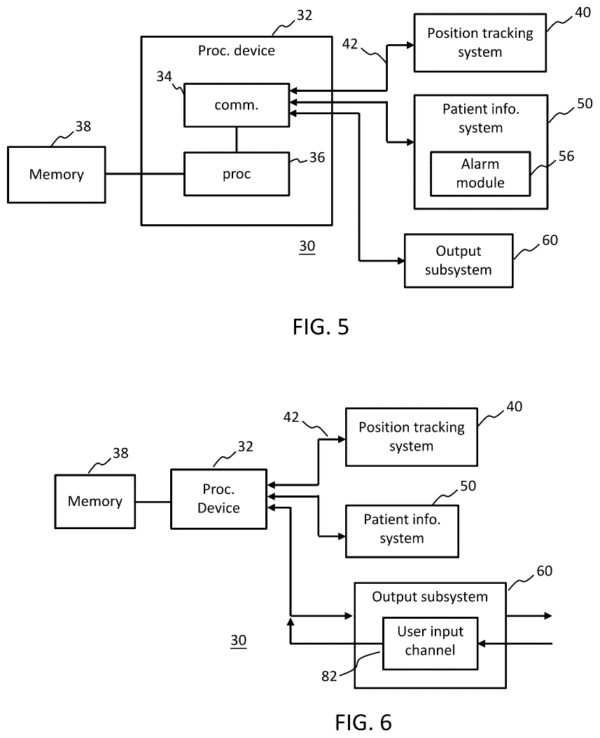

With reference to FIG. 5, in some embodiments, the patient information system 50 may include an alarm module 56 for generating alarms responsive to detection of predetermined clinical events related to patient status. An example implementation in which the patient information system 50 comprises an alarm module 56 is shown in FIG. 5. This feature is applicable to both the first aspect group (FIG. 1 and FIG. 2) and the second aspect group (FIG. 3 and FIG. 4).

The alarm module 56 of the patient information system 50 may generate alarms based on data received from one or more point-of-care medical devices comprised by the patient information system 50. For example, the alarm module 56 may be configured to continuously analyze data from the one or more point-of-care medical devices and compare this data against predefined thresholds or criteria to identify potential alarm conditions. The one or more point-of-care medical devices may include for example one or more patient monitors, ventilators, infusion pumps, and/or any other medical devices that collect and output patient data. The patient information system 50 may interface with various medical devices and sensors to collect real-time patient data. This data may include vital signs, medication administration records, laboratory results, and other clinical information relevant to patient care. For example, an alarm may be triggered if a patient's vital signs fall outside predetermined ranges or if a ventilator detects a problem with a patient's breathing.

When an alarm event is detected, the alarm module 56 may be configured to generate an alert signal that is transmitted to the processing device 32 through the communication module 34.

The alarm module 56 may in some examples be configured to categorize alarm events based on severity or urgency. This categorization allows the processing device 32 to prioritize alarm events and determine the most appropriate response.

With reference to FIG. 6, and as mentioned previously, in some embodiments, the user output subsystem 60 further comprises a user input channel 82 permitting a user to input a response to an alarm event. For example, the user input channel 82 may permit a user to input a response to acknowledge an alarm event. By way of further example, the user input channel 82 may permit a user to input a response to indicate current unavailability to attend to an alarm event. By way of further example, the user input channel 82 may permit a user to input a response to indicate a delegation instruction to delegate attendance to an alarm event to another clinical staff member. The user input channel may permit input by any of a plurality of clinical staff members, including the identified clinical staff member (assigned to the patient to which an alarm event relates), and also other clinical staff members. As noted above, in some embodiments, the system may be configured to detect input by at least one clinical staff member other than the identified clinical staff member indicative of acknowledgment of the alarm (or another indication related to the alarm), and wherein the system is configured to adapt the output of the information related to the alarm in dependence upon this input.

The user input channel 82 may be implemented in various forms, such as touchscreen interfaces on mobile devices, voice recognition systems, or physical buttons on wearable devices.

The user output subsystem may include one or more user interface devices, each comprising user input functionality, and wherein user inputs received at the user interfaces are coupled to the user input channel 82 of the user output subsystem 60. At least a subset of the user interfaces might be integrated in one or more user output devices comprised by the user output subsystem 60, or might be integrated in one or more point-of-care medical devices comprised by the patient information system 50.

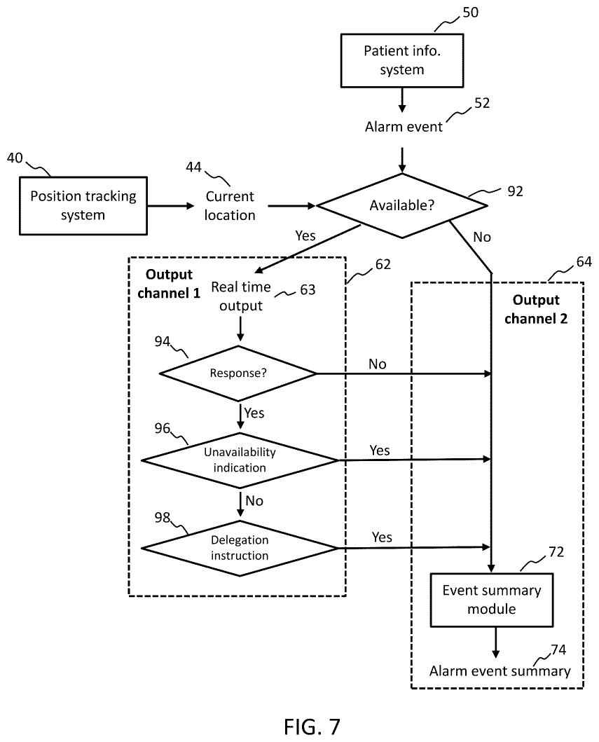

FIG. 7 depicts a flowchart illustrating one implementation of an example method which is consistent with the method and system of FIG. 1 and FIG. 2 as well as the method and system of FIG. 3 and FIG. 4.

As discussed previously, the patient information system 50 detects an alarm event 52 associated with a patient.

As also discussed previously, a position tracking system 40 detects a current location 44 of at least one clinical staff member.

Based on the location information 44, the method may comprise determining 92 an availability status of at least one clinical staff member assigned to the patient to which the alarm event relates.

If the clinical staff member is determined 92 to be available, the processing device 32 may use the first output channel 62 to provide a real time output 63 of information relating to the detected alarm to the at least one clinical staff member. In some examples, the real-time output may take the form of a real time alert indicating occurrence of the alarm. This may for example comprise generating an audible acoustic alert using an acoustic output device. This may be an acoustic output device at a location inside the medical unit. For example, the acoustic output device may be integrated in a point-of-care medical device. Additionally or alternatively, the real time output 63 may be an output of clinical information related to the alarm event. This may be output in real time via the first output channel 62 to the clinical staff member, for example via a mobile device associated with the clinical staff member, or via a display device.

If the clinical staff member is determined 92 to be unavailable, the processing device 32 is configured to instead output information relating to the detected alarm event using the second output channel 64. The second output channel permits communication of information about the alarm event to the at least one clinical staff member at a time after first occurrence of the alarm event. In the illustrated example, the second output channel 64 comprises use of an alarm event summary module 72 which is configured to generate alarm event summaries 74, wherein each alarm event summary 74 comprises information relating to at least one alarm event. The alarm event summary is communicated to the at least one clinical staff member using the user output subsystem 60. The user output subsystem may utilize various devices to communicate with clinical staff members. These devices may include smartphones, tablets, wearable devices, or stationary displays located throughout the healthcare facility.

Thus, in this particular example, the method comprises determining which output channel 62, 64 to use based on the detected location and availability of the assigned staff member, and wherein the first output channel 62 (providing real time output) is used when a staff member is determined to be available, and the second output channel 64 (providing post-hoc communication of an alarm event summary 74) is used when a staff member is determined to be unavailable.

If the clinical staff member is initially determined 92 to be available, following real time output 63 of alarm information to the at least one clinical staff member via the real time output channel 62, the method may further comprise one or more detection steps 94, 96, 98 for detecting one or more user inputs. The specific order of the detection steps is not critical. In one step 94, the method comprises detecting whether user has input a response to an alarm event through the user input channel 82 (see FIG. 6). If no response is detected, the method may further comprise additionally outputting information about the alarm by means of the second output channel 64. In the illustrated example, this may comprise generating an alarm event summary 74 for the alarm event.

The method may further comprise checking 96 for any user input at the user input channel by the at least one clinical staff member indicative of the clinical staff member being currently unavailable to attend to the alarm event. If unavailability is indicated via the user input channel, the method may further comprise additionally outputting information relating to the alarm event by means of the second output channel 64. In the illustrated example, this may comprise generating an alarm event summary 74 for the alarm event.

The method may further comprise checking 98 for any user input at the user input channel by the at least one clinical staff member of a delegation instruction. This comprises checking if the clinical staff member has input instructions to delegate the alarm event to another staff member. If a delegation instruction is detected 98, the method may further comprise additionally outputting information about the alarm by means of the second output channel 64. In the illustrated example, this may comprise generating an alarm event summary 74 for the alarm event.

Although not shown in FIG. 7, optionally, the method may comprise a further step of determining whether the identified clinical staff member is engaged in the care of a patient, and determining 92 the availability status as unavailable when engaged in care of another patient. This determination may be made using the current location of clinical staff member 44 provided by the position tracking system 40, in relation to known patient locations or designated patient care areas, or it may be made based on staff scheduling information stored in a database.

By implementing this decision process, the system 30 ensures that alarm information is communicated effectively to clinical staff members, taking into account their availability, location, and responses to previous alerts. This approach helps manage alarm fatigue and ensures that critical patient information reaches the appropriate staff members in a timely manner.

In the example of FIG. 7, the step 92 of detecting an availability status might instead be replaced by a step of selecting one or both of the first output channel 62 and/or the second output channel 64 based on the current location 44 of the at least one clinical staff member based on applying a location-based rule set or algorithm.

Embodiments of the invention described above employ a processing device. The processing device may in general comprise a single processor or a plurality of processors. It may be located in a single containing device, structure or unit, or it may be distributed between a plurality of different devices, structures or units. Reference therefore to the processing device being adapted or configured to perform a particular step or task may correspond to that step or task being performed by any one or more of a plurality of processing components, either alone or in combination. The skilled person will understand how such a distributed processing device can be implemented. The processing device includes a communication module or input/output for receiving data and outputting data to further components.

The one or more processors of the processing device can be implemented in numerous ways, with software and/or hardware, to perform the various functions required. A processor typically employs one or more microprocessors that may be programmed using software (e.g., microcode) to perform the required functions. The processor may be implemented as a combination of dedicated hardware to perform some functions and one or more programmed microprocessors and associated circuitry to perform other functions.

Examples of circuitry that may be employed in various embodiments of the present disclosure include, but are not limited to, conventional microprocessors, application specific integrated circuits (ASICs), and field-programmable gate arrays (FPGAs).

In various implementations, the processor may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

A single processor or other unit may fulfill the functions of several items recited in the claims.

The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

If the term “adapted to” is used in the claims or description, it is noted the term “adapted to” is intended to be equivalent to the term “configured to”.

Any reference signs in the claims should not be construed as limiting the scope.

1. A method for managing alarm events associated with each of one or more patients, wherein each of the one or more of patients is assigned to at least one clinical staff member, comprising:
detecting occurrence of an alarm event associated with a clinical status of a patient using a patient information system;
identifying at least one clinical staff member assigned to the patient;
detecting a current location of the identified at least one clinical staff member using data from a position tracking system, the position tracking system configured to track a location of the at least one clinical staff member;
determining an availability status of the identified at least one clinical staff member based at least in part on the location of the identified clinical staff member;
responsive to the availability status indicating that the identified clinical staff member is unavailable to attend to the alarm event:
generating an alarm event summary comprising information relating to the detected alarm event; and
communicating the alarm event summary to the identified clinical staff member using a user output subsystem.

2. The method of claim 1, wherein the method comprises determining an availability status indicating that the clinical staff member is available to attend to the alarm event when the clinical staff member is located in at least one predetermined area associated with clinical availability.

3. The method of claim 1, wherein the method comprises determining an availability status indicating that the clinical staff member is unavailable to attend to the alarm event when the clinical staff member is located in at least one predetermined area associated with clinical unavailability.

4. The method of claim 1,
wherein the user output subsystem further comprises a user input channel permitting a user to input a response to an alarm event;
wherein the method comprises detecting non-response of the identified clinical staff member to an alarm event, and
wherein, responsive to detecting non-response to the alarm event, the method further comprises generating an alarm event summary for the alarm event and communicating the alarm event summary to the identified clinical staff member using a user output subsystem.

5. The method of claim 1,
wherein the user output subsystem further comprises a user input channel permitting a user to input a response to an alarm event;
wherein the method comprises:
detecting an input by the identified clinical staff member at the user input channel indicating current unavailability to attend to the alarm event, and
responsive to detecting said input, generating an alarm event summary for the alarm event.

6. The method of claim 1,
wherein the user output subsystem further comprises a user input channel permitting a user to input a response to an alarm event;
wherein the method comprises:
detecting an input by the identified clinical staff member at the user input channel indicative of a delegation instruction, and
responsive to detecting said input, generating an alarm event summary for the alarm event.

7. The method of claim 1,
wherein the user output subsystem includes at least one mobile device associated with the identified clinical staff member assigned to the patient; and
wherein communicating the alarm event summary to the identified clinical staff member comprises communicating the alarm event summary to the at least one mobile device by means of a mobile communication interface.

8. The method of claim 1, wherein the alarm event summary includes information relating to clinical actions taken to resolve the alarm event, and optionally includes information relating to one or more required clinical actions.

9. The method of claim 1, wherein the alarm event summary includes information relating to a clinical status of the patient at a time of generating the alarm event summary and/or information relating to a clinical status of the patient at a time of communicating the alarm event summary to the clinical staff member.

10. The method of claim 1,
wherein the method comprises detecting multiple alarm events for a same patient during a period when the availability status of the identified at least one clinical staff member assigned to the patient indicates that the at least one clinical staff member is unavailable; and
generating a consolidated alarm event summary for the multiple alarm events.

11. The method of claim 1, further comprising:
detecting that the identified at least one clinical staff member has entered a predetermined area using data from the position tracking system; and
communicating the event summary to the identified at least one clinical staff member in response to detecting that the clinical staff member has entered the predetermined area.

12. The method of claim 1, wherein the method comprises determining, using data from the position tracking system, when the identified clinical staff member is engaged in the care of a patient, and determining the availability status of the clinical staff member as being indicative of unavailability to attend to the alarm event when the clinical staff member is engaged in the care of another patient.

13. A computer program product comprising computer program code configured, when executed by a processor, to cause the processor to perform a method in accordance with claims 1.

14. A processing device for managing alarm events associated with each of one or more patients, wherein each of the one or more patients is assigned to at least one clinical staff member, the processing device comprising:
one or more processors; and
a communication module;
wherein the communication module is communicatively coupled with:
a position tracking system configured to track a location of the at least one clinical staff member, and
a patient information system configured to detect alarm events associated with a clinical status of each of the one or more patients;
wherein the one or more processors are configured to:
detect occurrence of an alarm event associated with a patient using the patient information system;
identify at least one clinical staff member assigned to the patient;
determine an availability status of the identified at least one clinical staff member based at least in part on the location of the identified clinical staff member;
responsive to the availability status indicating that the identified clinical staff member is unavailable to attend to the alarm event:
generate an alarm event summary comprising information relating to the detected alarm event; and
communicate the alarm event summary to the identified clinical staff member using a user output subsystem.

15. A system for managing alarm events associated with each of one or more patients, wherein each of the one or more patients is assigned to at least one clinical staff member, comprising:
a position tracking system configured to track a location of the at least one clinical staff member;
a patient information system configured to detect alarm events associated with a clinical status of each of the one or more patients; and
a processing device as claimed in claim 14.
